(19) United States
(12) Patent Application Publication
(10) Pub. No.: US 2025/0213304 A1
(43) Pub. Date: Jul. 3, 2025

(54) MARKER BAND LOCKING FEATURE

(71) Applicant: KONINKLIJKE PHILIPS N.V., EINDHOVEN (NL)

(72) Inventor: Kylie Cyrielle FORD, COLORADO SPRINGS, CO (US)

(21) Appl. No.: 18/850,129

(22) PCT Filed: Mar. 29, 2023
(86) PCT No.: PCT/EP2023/058073

§ 371 Date: Sep. 24, 2024

Related U.S. Application Data
(60) Provisional application No. 63/325,649, filed on Mar. 31, 2022.

(51) Int. Cl. A61B 18/24; A61B 18/00; A61B 18/22; A61M 25/00; A61M 25/01

(52) U.S. Cl. CPC A61B 18/245; A61M 25/0009; A61B 2018/0041; A61B 2018/00577; A61B 2018/2211; A61M 25/0108

(57) ABSTRACT

A laser catheter device (10) comprises an inner tube (12) defining a lumen (14). the inner tube having a central axis; a ring of optical fibers (16) surrounding the inner tube and the inner lumen, the optical fibers oriented parallel with the central axis; a jacket (18) surrounding the ring of optical fibers and secured to the ring of optical fibers; an annular marker (20) disposed over an end of the ring of optical fibers; and a metal strip or wire (22. 26) oriented parallel with the central axis and secured to a portion of the inner tube. the metal strip or wire having a distal end (24, 27) attached to the annular marker.

### FIELD

The following relates generally to the vascular arts, catheter sensing arts, imaging arts, radiopaque marker arts, and related arts.

### BACKGROUND

Existing laser catheters include a ring of optical fibers surrounding an inner lumen which may serve as the guidewire lumen, aspiration channel, or other function. An inner polytetrafluoroethylene (PTFE) or other plastic layer defines the wall of the inner lumen, and an outer PTFE or other plastic jacket (e.g., Hytrel® or Pebax®) is heat fused to the outside of the optical fiber ring. Additionally, a mandrel (i.e., a stiffening rod) having a length of typically about 150 cm and occupies a majority of the length of the catheter. A stiffer end of the mandrel terminates within a tail tube portion of the catheter, and a tapered end of the mandrel terminates in between a port and the tip. The port is where the inner lumen terminates at about 10 cm from the tip is included near (but not at) the distal end of the laser catheter, to provide stiffening near the operative end. The stiffening rod is typically a stiff stainless steel rod or wire that runs along the axis of the fiber ring, and is secured with the outer surface of the fiber ring along with the outer PTFE jacket by a fusing process.

In existing designs, a tapered marker band of a radiopaque material such as a platinum-iridium alloy is disposed over the end of the optical fiber band to serve as a marker during fluoroscopic or computed tomography (CT) imaging used to provide visual guidance to medical personnel performing an intravascular procedure such as laser ablation of a clot using the laser catheter. The taper of the tapered band together with epoxy is applied to the inside of a marker band to hold the band, fibers, and inner lumen together. Once this process is completed, and the epoxy is cured, the jacket is fused to the fibers and the band.

The following discloses certain improvements to overcome these problems and others.

### SUMMARY

In some embodiments disclosed herein, a laser catheter device comprises an inner tube defining a lumen, the inner tube having a central axis; a ring of optical fibers surrounding the inner tube and the inner lumen, the optical fibers oriented parallel with the central axis; a jacket surrounding the ring of optical fibers and secured to the ring of optical fibers; an annular marker disposed over an end of the ring of optical fibers; and a metal strip or wire oriented parallel with the central axis and secured to a portion of the inner tube, the metal strip or wire having a distal end attached to the annular marker.

In some embodiments disclosed herein, a method of manufacturing a laser catheter device includes: forming an assembly including an inner tube defining a lumen, the inner tube having a central axis, and a ring of optical fibers surrounding the inner tube and the inner lumen, the optical fibers oriented parallel with the central axis; adding an end assembly including an annular marker disposed over an end of the assembly and a metal strip or wire oriented parallel with the central axis and having a distal end attached to the annular marker; and securing a plastic jacket over the assembly including securing the plastic jacket to the ring of optical fibers and to the annular marker by a heat fusing process.

In some embodiments disclosed herein, a laser catheter comprises a tube defining an inner lumen, the inner tube having a central axis; a ring of optical fibers surrounding the inner lumen, the optical fibers oriented parallel with the central axis; a jacket surrounding the ring of optical fibers and secured to the ring of optical fibers; an annular marker disposed over an end of the ring of optical fibers; and the annular marker includes a textured outer surface, and the jacket is secured to the textured portion.

One advantage resides in providing a catheter with an improved connection of a marker band to an optical fiber ring.

Another advantage resides in improving visualization of a catheter undergoing imaging.

Another advantage resides in adding an additional flexible wire to a catheter to improve a connection between a marker band and an optical fiber ring.

Another advantage resides in roughening a texture of a marker band to improve a connection between the marker band and an optical fiber ring of a catheter.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a laser catheter device in accordance with the present disclosure.

FIGS. 2 and 3 illustrate other embodiments of the device of FIG. 1.

FIG. 4 diagrammatically illustrates a method of manufacturing the device of FIG. 1.

### DETAILED DESCRIPTION

The following discloses various approaches for improving the attachment of the marker band to the tip of the laser catheter. In a first approach, the stiffening rod is extended in length toward the distal end of the catheter (and/or shifted toward the distal end of the catheter) so that the distal end of the stiffening rod overlaps the marker band. The inner surface of the marker band is soldered or otherwise attached to the overlapping end of the stiffening rod. The marker band may optionally still be tapered.

In a variant second approach, the stiffening rod is not moved or extended. Instead, an additional wire is included, which has one end soldered or otherwise attached to the stiffening rod and the other end overlapping the marker band and soldered or otherwise attached to the inner surface of the marker band. This provides additional design flexibility since the flexibility of the additional wire may be different from (for example, more flexible than) the stiffening rod.

In a third approach, the outer surface of the marker band is roughened or textured, for example by etching, sanding, sand blasting, or the like. The outer jacket, made from, for example, Pebax® (available from Arkema S.A., Colombe, France), is then formed to go over the roughened outer surface of the marker band (rather than going up to the marker band as in the existing design). The jacket is heat-fused to the catheter, and the jacket would be placed over the band so that the texturing could provide grip to hold the jacket to the band as it is fused down. Epoxy or other adhesive could be used to help with this fuse process. The advantage of having the jacket secured to the marker band is that it is another point of contact besides the epoxy inside the marker band to hold it on the catheter.

The third approach can be used in combination with either of the first or second approaches.

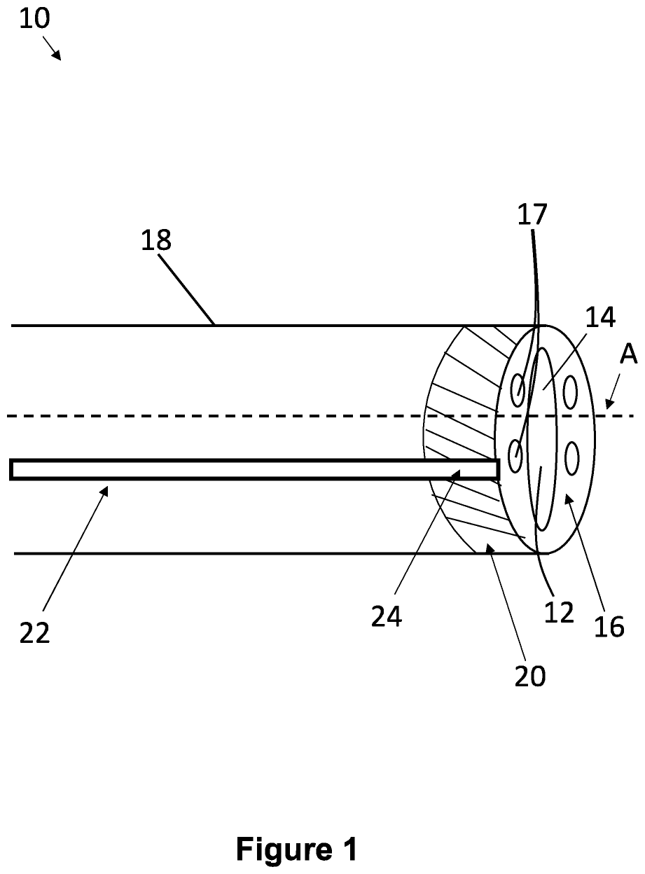

With reference to FIG. 1, a distal end of an illustrative laser catheter device 10 is diagrammatically shown in a cut-away view. As shown in FIG. 1, the laser catheter device 10 includes an inner tube 12 defining a lumen 14 and having a central axis A. In some embodiments, the inner tube 12 comprises a plastic (e.g., PTFE). A ring of optical fibers 16 (four of which are shown in FIG. 1) surrounds the inner tube 12 and the inner lumen 14. The optical fibers are oriented parallel with the central axis A and run the length of the catheter. The distal ends 17 of the optical fibers (an illustrative four of which are shown in FIG. 1) serve as the laser light output aperture. The distal ends 17 may optionally be shaped by heat treatment or otherwise modified to modify the optical properties of the laser light output aperture. The proximal end (not shown) of the ring of optical fibers 16 suitably terminate at an optical coupler designed to connect with a laser light source such as an excimer laser (not shown). The ring of optical fibers 16 surrounding the inner tube 12 form an assembly.

A jacket 18 surrounds the ring of optical fibers 16, and is secured to the ring of optical fibers 16 to hold the assembly together. In some embodiments, the jacket 18 comprises a plastic (e.g., Pebax®) and is secured to the ring of optical fibers 16 by heat fusion. An annular marker 20 (shown with single cross-hatching in FIG. 1) is disposed over an end of the ring of optical fibers 16 and attached to the ring of optical fibers 16 with epoxy. In some embodiments, the annular marker 20 comprises a radiopaque material such as a platinum-iridium alloy.

FIG. 1 also shows a metal strip or wire 22 oriented parallel with the central axis A and to a proximal end of the inner tube 12. The metal strip or wire 22 is free-floating at a distal end 24 thereof adjacent the marker 20. In some embodiments, the metal strip or wire 22 comprises stainless steel, although other materials are contemplated. Such a metal strip or wire 22 serves to stiffen the proximal end of the catheter to facilitate maneuvering it through the vasculature to position the distal end proximate to a blood clot or other build-up within the blood vessel that is to be ablated by laser light delivered via the output aperture of the ring of optical fibers 16. The metal strip or wire 22 thus serves as a stiffener, and is also sometimes referred to as a mandrel in the art. In a common design, the stiffener 22 is about 150 cm long, although a longer or shorter stiffener 22 is also contemplated, and the stiffener in this common design does not extend all the way to the tip of the distal end of the catheter.

The metal strip or wire 22 in the embodiment of FIG. 1 differs from this common design in that it has a distal end 24 that extends close to or all the way to the tip of the distal end of the catheter, and is attached to the annular marker 20. For example. an inner surface of the annular marker 20 is attached to the distal end 24 of the metal strip or wire 20 (e.g., by soldering). In this way, the soldered connection of the annular marker 20 to the distal end 24 of the stiffener 22 serves to improve the attachment of the marker band 20 to the tip of the laser catheter.

In the embodiment shown in FIG. 1, the metal strip or wire 22 comprises the stiffening rod of the distal end of the laser catheter device 10, which is operative to stiffen the portion of the ring of optical fibers 16 coinciding with the stiffening rod 22 along the central axis A. The distal end 24 of the stiffening rod 22 is attached to the annular marker 20 to serve the secondary purpose of improving attachment of the marker band 20 to the tip of the laser catheter device 10.

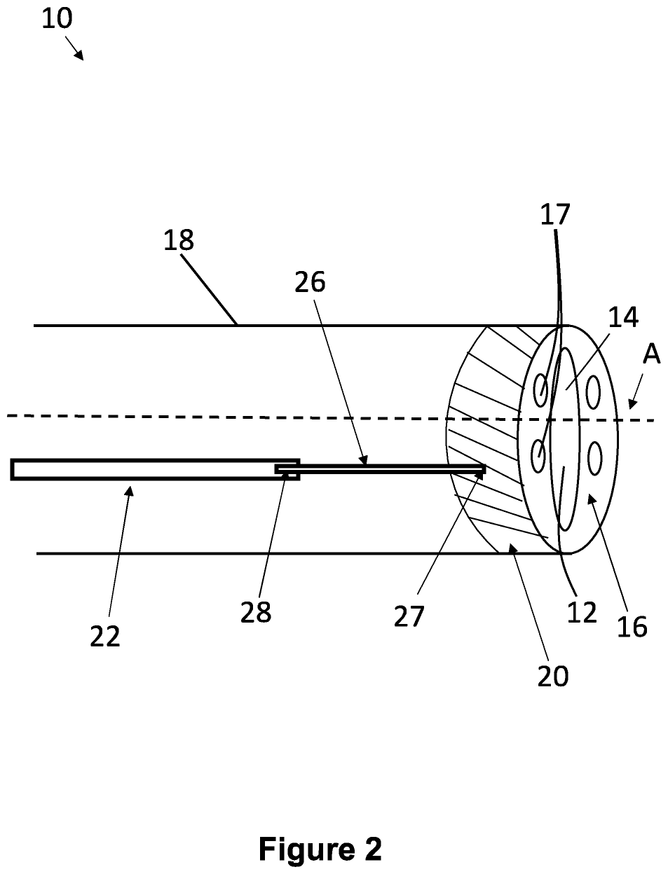

In a second embodiment, as shown in FIG. 2, the stiffening rod 22 is not extended to the tip of the laser catheter device 10, and its distal end is not attached to the annular marker 20. Instead, a separate metal strip or wire 26 is added. A distal end 27 of the added metal strip or wire 26 is attached to the annular marker 20, for example by soldering, and a proximal end 28 of the added metal strip or wire 26 opposite the distal end 27 is attached to the stiffening rod 22, for example by soldering. In some embodiments, the added metal strip or wire 26 is more flexible than the stiffening rod 22, which may provide benefit in manipulation of the tip of the laser catheter. In some embodiments, the added metal strip or wire 26 is made of stainless steel, although other materials are contemplated.

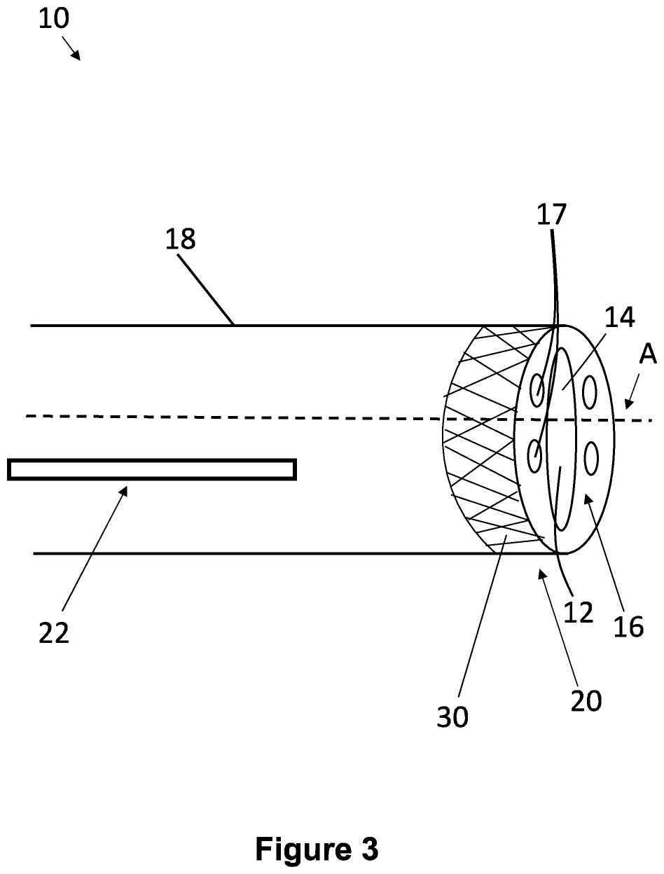

In a third embodiment, as shown in FIG. 3, the annular marker 20 includes a textured outer surface 30 (shown with double cross-hatching), and the jacket 18 is secured to the textured portion. In this embodiment, the outer jacket 18 is disposed over (at least a portion of) the textured outer surface 30 of the annular marker 20, and the heat fusing process of the outer jacket 18 to the ring of optical fibers 16 also operates, together with the texturing, to heat fuse the outer jacket to the textured outer surface 30 of the annular marker 20, thereby improving the attachment of the marker band 20 to the tip of the laser catheter. In the embodiment of FIG. 3, the stiffening rod 22 does not extend to the tip of the catheter (as in the embodiment of FIG. 1), and the added metal strip or wire 26 of the embodiment of FIG. 2 is also not included in the embodiment of FIG. 3. Hence, in the illustrative embodiment of FIG. 3 the improved attachment of the annular marker 20 is only by way of the textured outer surface 30 being heat fused to the distal end of the outer jacket 18. However, this approach shown in FIG. 3 can also be used in the embodiment of FIG. 1 or the embodiment of FIG. 2 in combination. That is, the textured portion 30 can also be added to the embodiments shown in FIGS. 1 and 2. In such combined embodiments, the attachment of the annular marker 20 is improved both by way of the textured outer surface 30 being epoxy fused to the distal end of the outer jacket 18 and by the soldered or other attachment of the distal end 24, 27 of the metal strip or wire 22 or 26 (referring to the embodiment of FIG. 1 or FIG. 2, respectively).

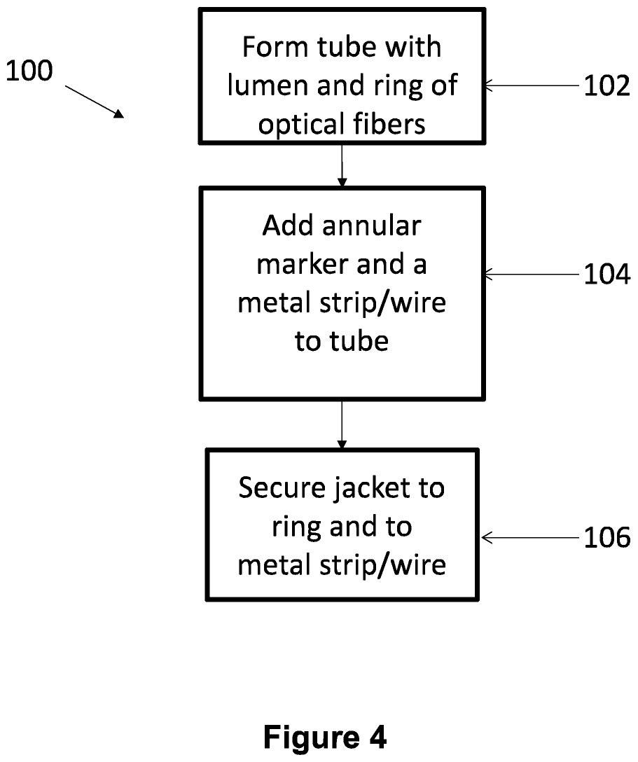

Referring to FIG. 4, and with continuing reference to FIGS. 1-3, an illustrative embodiment of manufacturing method 100 to manufacture the laser catheter device 10 is diagrammatically shown as a flowchart. At an operation 102, an assembly including the inner tube 12 defining the lumen 14 and the surrounding ring of optical fibers 16 is formed. The inner tube 12 has the central axis A. The ring of optical fibers 16 surrounds the inner tube 12 and the inner lumen 14. The optical fibers 16 are oriented parallel with the central axis A.

At an operation 104, an end assembly including an annular marker 20 disposed over an end of the tube 12, and the stiffening rod 22 oriented parallel with the central axis A and having a distal end 24 is attached to the annular marker 20. For manufacturing the embodiment of FIG. 1, the stiffening rod 22 is operative to stiffen the portion of the ring of optical fibers 16 coinciding with the stiffening rod 22 along the central axis A, and the operation 104 includes attaching the distal end 24 of the stiffening rod 22 to the annular marker 20 by soldering or the like.

Alternatively, for manufacturing the embodiment of FIG. 2, the operation 104 includes adding the stiffening rod 22 and also adding the added metal strip or wire 26 with one end 28 soldered or otherwise attached to the stiffener 22 and the other end soldered or otherwise attached to the annular marker 20.

At an operation 106, a plastic (e.g., Pebax®) jacket 18 is secured over the assembly including securing the plastic jacket 18 to the ring of optical fibers 16 and to the annular band 20 by a heat fusing process. In manufacturing the embodiment of FIG. 3, the operation 106 also operates to secure the end of the jacket 18 to the textured outer surface 30 of the annular marker 20.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A laser catheter device, comprising:
an inner tube defining a lumen, the inner tube having a central axis;
a ring of optical fibers surrounding the inner tube and the inner lumen, the optical fibers oriented parallel with the central axis;
a jacket surrounding the ring of optical fibers and secured to the ring of optical fibers;
an annular marker disposed over an end of the ring of optical fibers; and
a metal strip or wire oriented parallel with the central axis and secured to a portion of the inner tube, the metal strip or wire having a distal end attached to the annular marker.

2. The laser catheter device of claim 1, wherein the metal strip or wire comprises a stiffening rod operative to stiffen the portion of the ring of optical fibers coinciding with the stiffening rod along the central axis, wherein the distal end of the stiffening rod is attached to the annular marker.

3. The laser catheter device of claim 1, further comprising:
a stiffening rod operative to stiffen the portion of the ring of optical fibers coinciding with the stiffening rod along the central axis;
wherein the metal strip or wire is different from the stiffening rod and the distal end of the metal strip or wire is attached to the annular marker and a proximal end of the metal strip or wire opposite the distal end is attached to the stiffening rod.

4. The laser catheter device of claim 3, wherein the metal strip or wire is more flexible than the stiffening rod.

5. The laser catheter device of claim 1, wherein an inner surface of the annular marker is attached to the distal end of the metal strip or wire.

6. The laser catheter device of claim 5, wherein the inner surface of the annular marker is soldered to the distal end of the metal strip or wire.

7. The laser catheter device of claim 1, wherein the annular marker includes a textured outer surface, and the jacket is secured to the textured portion.

8. The laser catheter device of claim 1, wherein the jacket comprises a plastic and is secured to the ring of optical fibers and the annular marker by heat fusion.

9. The laser catheter device of claim 1, wherein the metal strip or wire comprises stainless steel.

10. The laser catheter device of claim 1, wherein the annular marker comprises a radiopaque material.

11. The laser catheter device of claim 10, wherein the marker comprises a platinum-iridium alloy.

12. The laser catheter device of claim 1, wherein the inner tube comprises plastic.

13. A method of manufacturing a laser catheter device, the method comprising:
forming an assembly including an inner tube defining a lumen, the inner tube having a central axis, and a ring of optical fibers surrounding the inner tube and the inner lumen, the optical fibers oriented parallel with the central axis;
adding an end assembly including an annular marker disposed over an end of the assembly and a metal strip or wire oriented parallel with the central axis and having a distal end attached to the annular marker; and
securing a plastic jacket over the assembly including securing the plastic jacket to the ring of optical fibers and to the annular marker by a heat fusing process.

14. The method of claim 13, wherein the plastic jacket comprises a Pebax® jacket.

15. The method of claim 13, wherein the metal strip or wire comprises a stiffening rod operative to stiffen the portion of the ring of optical fibers coinciding with the stiffening rod along the central axis, wherein the distal end of the stiffening rod is attached to the annular marker.

16. The method of claim 13, wherein the end assembly further includes a stiffening rod operative to stiffen the portion of the ring of optical fibers coinciding with the stiffening rod along the central axis;
wherein the metal strip or wire is separate from the stiffening rod and the distal end of the metal strip or wire is attached to the annular marker and a proximal end of the metal strip or wire opposite the distal end is attached to the stiffening rod; and
wherein the epoxy fusing process also secures the plastic jacket to the annular marker.

17. A laser catheter device, comprising:
a tube defining an inner lumen, the inner tube having a central axis;
a ring of optical fibers surrounding the inner lumen, the optical fibers oriented parallel with the central axis;
a jacket surrounding the ring of optical fibers and secured to the ring of optical fibers; and
an annular marker disposed over an end of the ring of optical fibers;
wherein the annular marker includes a textured outer surface, and the jacket is secured to the textured portion.

18. The laser catheter device of claim 17, further comprising:
a metal strip or wire oriented parallel with the central axis and secured to the jacket, the metal strip or wire having a distal end attached to the annular marker.

19. The laser catheter device of claim 18, wherein the metal strip or wire comprises stainless steel.

20. The laser catheter device of claim 17, wherein the jacket comprises a plastic and is secured to the ring of optical fibers by heat fusion.
